(19) United States
(12) Patent Application Publication
(10) Pub. No.: US 2012/0309753 A1
(43) Pub. Date: Dec. 6, 2012

(54) METHOD OF DISSOLVING ANTIFUNGAL AGENT, AND COMPOSITIONS WITH A HIGH CONCENTRATION OF ANTIFUNGAL AGENT, SUITABLE FOR APPLICATION TO THE NAIL

(75) Inventors: Claire Mallard, Mougins (FR); Nathalie Willcox, Magagnosc (FR)

(73) Assignee: GALDERMA RESEARCH & DEVELOPMENT, Biot (FR)

(21) Appl. No.: 13/254,989

(22) PCT Filed: Mar. 5, 2010
(86) PCT No.: PCT/EP2010/052817

§ 371 Date: Oct. 10, 2011

Related U.S. Application Data
(60) Provisional application No. 61/202,516, filed on Mar. 6, 2009.

(51) Int. Cl. A61K 31/5375; A61P 31/00; A61K 31/137; A61P 31/10

(52) U.S. Cl. 514/231.2; 514/655

(57) ABSTRACT

A pharmaceutical composition is described which includes: an antifungal agent from the class of the allylamines or the morpholines, at a concentration of at least 5%; a ternary solvent system comprising water; at least one branched or straight chain C2-C8 alkanol; at least one glycol; the total amount of water representing more than 30% (w/w) of the composition. This composition is intended for application to the nail for the treatment of onychomycoses.

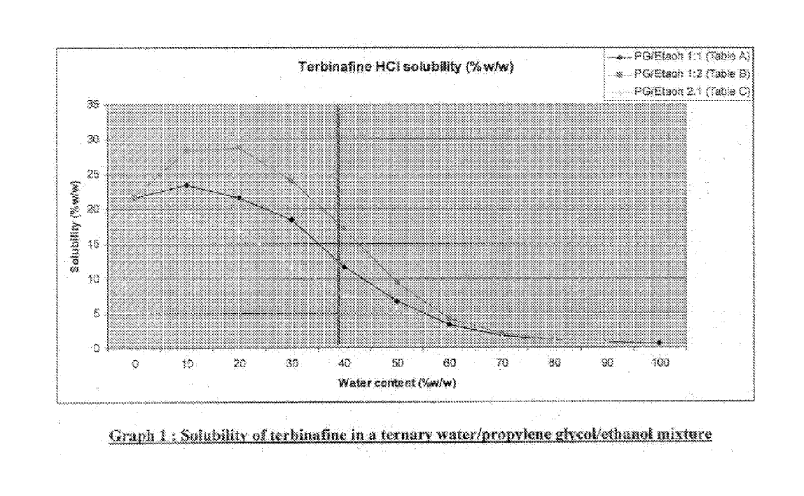

### TECHNICAL BACKGROUND

The present invention relates to the development of a ternary solvent system comprising water that allows substantial amounts of antifungal agents from the class of the allylamines or from the class of the morpholines to be dissolved. This invention allows compositions to be prepared that are specifically adapted to application to the nail, the latter possibly being optionally perforated or pretreated chemically or physically.

Pharmaceutical or dermatological compositions of this kind are useful especially for the treatment, in humans and animals, of onychomycoses, especially those due to dermatophytes or to Candida.

### PRIOR ART

Antifungal agents from the class of the allylamines, especially terbinafine or naftifine, and those from the class of the morpholines, especially amorolfine, are promising compounds in antifungal control. Their presumed or demonstrated mode of action involves inhibition at the level of ergosterol, a specific constituent of the wall of fungal cells, especially via the inhibition of squalene epoxidase (“Terbinafine: Mode of action and properties of the squalene epoxidase inhibition” British Journal of Dermatology, Volume 126 Issue s39, pages 2-69 (February 1992); “Preclinical data and mode of action of amorolfine”, Dermatology, 1992, vol. 184, SUP1 (10 ref.), pp. 3-7).

Traditionally, antifungals have been administered either topically or orally.

By the oral route, terbinafine administered at a level of 250 mg/day for 6 months has proved to be effective and of low toxicity in the treatment of onychomycoses due to Trichophyton rubrum. However, treatment of this kind raises a problem of duration and of cost, and sometimes has side-effects affecting the digestive sphere, taste disorders or else transitory skin eruptions (GUPTA A, LYNDE C, LAUZON G et al. Cutaneous adverse effects associated with terbinafine therapy: 10 case reports and a review of the literature. Br J Dermatol, 1998, 138: 529-532).

By the topical route, transungual administration represents an alternative solution to oral administration for the treatment of onychomycoses. However, the problem which arises in the case of administration to the nail is to ensure the penetration and spreading of the antifungal agents in the nail, allowing therapeutically effective concentrations to be attained in the nail and under the nail, in other words in the nail bed.

The nail as such is composed essentially of keratin, a fibrous protein which is indeed insoluble yet which exhibits an affinity for water. Thus the nail is considered to be hydrophilic in nature, which is to say that it behaves as a hydrogel. Consequently, and for transungual application, it would appear mandatory to formulate the antifungal agent in water. Water, however, has difficulties in its compatibility with the aforementioned antifungal agents of interest, especially terbinafine, which per se exhibit little spreading ability into the nail, and, moreover, are virtually insoluble in water.

Among all of the technical solutions proposed for transungual formulations of antifungal agents in the prior art, the solution used has been that of an aqueous-alcoholic mixture with addition of polymers.

Alternatively, document EP 0 503 988 proposed admixing the aqueous-alcoholic medium with hydrophilic penetrants, which were hitherto known and used for promoting the transcutaneous penetration of active agents through the horny layer of the skin; this horny layer is lipophilic in nature and behaves as a water barrier.

However, even in the presence of such hydrophilic penetrants, the proportion of water in the compositions described in document EP 0 503 988 is never greater than 30% of the total weight of the composition.

There is therefore a need to find new formulations for antifungal agents that allow improved spreading through the nail of an effective amount of active principle.

### SUMMARY OF THE INVENTION

The present invention lies in the demonstration of a ternary solvent system which comprises water and which combines a capacity for substantial amounts of antifungal agents of interest to be dissolved, the possibility of a large amount of water, and a capacity for effective spreading and effective penetration into and through the keratinized ungual tablet of the nail.

By “large amount of water” is meant a total amount of water in the composition of more than 30% by weight, relative to the total weight of the composition.

More specifically, the present invention provides a dermatological composition intended for the treatment of onychomycoses, which takes the form of a solution and comprises:
first, an effective amount of at least one antifungal agent;
second, a solvent medium for said antifungal agent (or agents) that is composed of a mixture of water, at least one branched or straight chain C2-C8 alkanol, and at least one glycol that has free hydroxyl functions.

A composition of this kind is characterized in that the total water represents more than 30% by weight, relative to the total weight of the composition (w/w), advantageously more than 33%, or even more than 35%, or, indeed, even more than 40%.

In the context of the invention, an “effective amount” of at least one antifungal agent is a substantial amount of said agent in the composition. It is obvious that the problem of solubility in the solvent medium, more particularly in water, arises for substantial amounts of this kind.

In practice, the substantial amount of antifungal agent, as will be defined below, represents more than 5%, or even at least 8%, or, indeed, even at least 10% (w/w) of the total composition. Hence it is possible to envisage up to 15% or even 20% of this agent in the composition.

It is of course possible to contemplate a mixture of antifungal agents, possibly from different classes.

As already stated, the antifungal agents of interest that are more particularly a target of the present invention are those from the class of the allylamines and from the class of the morpholines, the class of the allylamines being preferred.

Within the class of the allylamines, mention may be made, in particular, of terbinafine and its acid salts, and naftifine and its acid salts. Among the acid salts, preference will be given to terbinafine hydrochloride and naftifine hydrochloride, the respective formulae of which are as follows:

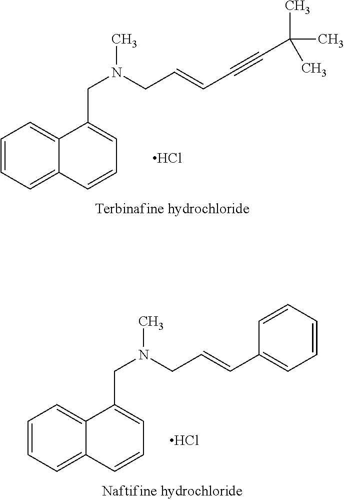

Among the molecules from this class, preference is given to terbinafine.

Alternatively, the antifungal agent may belong to the class of the morpholines, especially amorphine and its acid salts, for which similar problems of solubility in water arise.

As indicated above, the invention is based on the demonstration of a ternary solvent system comprising water and exhibiting a synergy in the dissolution of the antifungal agents of interest.

This system comprises:
water, which is known to be a very poor solvent of antifungal agents from the class of the allylamines or from that of the morpholines;
a short chain alcohol, and more specifically, at least one branched or straight chain C2-C8 alkanol, these alcohols being known to be solvents for antifungal agents from the class of the allylamines or from that of the morpholines;
at least one glycol.

A very poor solvent is a solvent which allows dissolution of not more than 1% (w/w) of the antifungal agent from the class of the allylamines or from that of the morpholines.

Hence, remarkably, the inventors have demonstrated that this combination of three solvents comprising water exhibits a synergy in the dissolution of the antifungal agents of interest, thereby allowing the amount of water in the mixture to be increased while at the same time allowing the dissolution of substantial amounts of antifungal agents.

As already stated, and remarkably, the total water represents more than 30% by weight, relative to the total weight of the composition (w/w), advantageously more than 33%, or even more than 35% or, indeed, even more than 40%. Consequently, the total water advantageously represents more than a third of the ternary solvent mixture.

By total water is meant the amount of water introduced as such into the composition, with the addition of the amount of water originating from the various solvents and/or excipients in the composition, where they contain water.

On the other hand, the total water represents advantageously less than 60%, or even less than 50%, of the total composition.

The second entity in this ternary system is a short chain alcohol, and more specifically at least one branched or straight chain C2-C8 alkanol, preferably ethanol, isopropanol and n-butanol. Ethanol is particularly preferred. A mixture of different alcohols may also be contemplated.

Lastly, this ternary solvent system comprises at least one glycol. A glycol here is a compound which has at least two hydroxyl functions. The invention relates more particularly to glycols whose two hydroxyl functions are free, which is to say that they are not engaged in an ether or ester linkage. Such glycols include, for example, propylene glycol, butylene glycol, hexylene glycol, ethylene glycol and polyethylene glycols. Propylene glycol is preferred. A mixture of different glycols may also be contemplated.

Advantageously, the ternary solvent system represents at least 60%, or even 70%, 80%, or, indeed, even 90% (w/w) of the total composition.

Moreover, and advantageously, the proportion of alcohol is greater than or equal to that of glycol.

Even more advantageously, the total proportion of water is greater than that of glycol.

Generally speaking, a pharmaceutical or dermatological composition according to the invention takes the form of a solution. It may equally be formulated as a lotion, spray, emulsion, foam or gel, which advantageously is fluid.

The composition according to the invention may also include at least one additive selected from the group consisting of:
preservatives, such as phenylethyl alcohol, benzyl alcohol and phenoxyethanol, parabens and derivatives;
antioxidants, such as butylated hydroxyanisole (BHA), butylated hydroxytoluene (BHT), palmityl ascorbate, alpha-tocopherol and/or its esters;
dyes, fillers or pigments, such as the titanium micas commonly used in the cosmetics field for producing nail varnish;
polymers capable of preventing the flow of the composition before penetration, such as, for example, alkylcellulose derivatives, especially methylcelluloses, ethylcelluloses and hydroxy-alkylcelluloses, such as those sold under the name “KLUCEL”;
chelating agents such as disodium edetate (EDTA);
emollients such as cyclomethicone;
antiseptics, especially acetic acid or chlorhexidine.

The amounts of each of these additives are readily determined by a person skilled in the art.

As already stated, a composition according to the invention is particularly adapted for the treatment of onychomycoses transungually. Hence it is intended for application to the surface of the nail.

### WORKING EXAMPLES

The invention and its attendant advantages will emerge more clearly from the working examples which follow. In no case, however, are these examples limitative.

In these examples, the following compounds were tested:
antifungal agent=terbinafine hydrochloride (terbinafine HCl);
water=purified water;
alcohol=ethanol;
glycol=propylene glycol.

1/Demonstration of Synergy of the Ternary Solvent System for the Dissolution of the Antifungal Agent:

Different solubility tests on terbinafine HCl were carried out in different solvent systems. The solubility was both estimated visually by Method 1 below, then quantified by HPLC assay by Method 2 below.

### Method 1: Dissolution of the Terbinafine HCl

Terbinafine HCl is weighed out accurately into a 20 ml flask and then the solvents are added. The flask is subsequently placed, with magnetic stirring, in a chamber thermostated at 20° C., for 16 h. If some of the active ingredient has not dissolved, a further addition of solvent is made. The flask is then placed again, with magnetic stirring, in the thermostated chamber, for 16 h, until dissolution is complete. If this is not so, the preceding step is repeated.

### Method 2: Quantification of the Terbinafine by HPLC

### Preparation of Solutions:

### Diluent:

| Solvent | Acetonitrile | Ultrapure water |
|---|---|---|
| Volume | 50 | 50 |

### Stock Solutions

Solution A (300 μg/ml)

Weigh out precisely approximately 15 mg of terbinafine HCl into a 50 ml volumetric flask. Dissolve in the diluent and make up to the volume with the same solvent. Prepare the solution in duplicate (A1, A2)

Daughter Solution (60 μg/ml)

Dilute solution A to 1/5 in the diluent. Dilution

A1 [symbol] STD1, dilution A2 [symbol] STD2

### Standard Range

The standard range is produced by varying the injection volume of solution A, at an equivalent concentration of 4 μg/ml to 120 μg/ml.

### Sample Solutions

2 tests per sample are prepared, of concentrations from 40 μg/ml to 100 μg/ml.

### Operating Conditions

### Eluent Phase

| | solvents | | | |
|---|---|---|---|---|
| | Acetonitrile | Methanol | Water | Triethylamine |
| Mixtures | (ml) | (ml) | (ml) | (ml) |
| Path A | 99 | 0 | 0 | 1 |
| Path B | 0 | 100 | 0 | 0 |
| Path C | 0 | 0 | 99 | 1 |

### Parameters of the Chromatography System

Column: Sun Fire C18 4.6×100 mm 3.5 μm, fitted with a precolumn.
Column temperature: 30° C.
Flow rate: 1.5 ml/min
Injected volume: 30 μl for the sample solutions
Analysis time: 10 min
Detection at: 285 nm
Gradient:

| Time (min) | Path A (%) | Path B (%) | Path C (%) |
|---|---|---|---|
| 0 | 60 | 20 | 20 |
| 7 | 80 | 20 | 0 |
| 9 | 80 | 20 | 0 |
| 9.1 | 60 | 20 | 20 |
| 10 | 60 | 20 | 20 |

### Chromatography System Conformity Test

Inject the control solution STD 6 times, and, on the peak of the assayed active ingredient, measure the following parameters:

| Parameter: | Limit: | Tolerance: |
|---|---|---|
| Retention time: | approximately: | +/−1 min |
| | 5.73 min | |
| Repeatability of areas: | <2% | — |
| CV (%/mean): | | |
| Peak symmetry: | 0.8 to 1.4 | |
| Number of theoretical | >10 000/column | |
| plates (mean): | | |

Inject a blank sample (diluent) and check that no peak of the blank sample interferes with the terbinafine HCl.
Inject solutions STD1 and STD2 and check that the % deviation between the 2 controls is <2%.
   A/ Binary solvent system:

A-1/Ethanol/Propylene Glycol
In ethanol, the visual solubility of the terbinafine HCl is 19.7% w/w, confirmed at 18.09% w/w by HPLC assay;
In propylene glycol, the visual solubility of the terbinafine HCl is 8.9% w/w, confirmed at 10.3% w/w by HPLC assay;
In a 50/50 ethanol/propylene glycol mixture, the visual solubility of the terbinafine HCl is 23.3% w/w, confirmed at 20.4% w/w by HPLC assay.

In conclusion, although propylene glycol is not as good a solvent of the terbinafine HCl as is ethanol, the solubility obtained in the binary mixture is slightly greater than that obtained in ethanol. By additive effect, the expected solubility of the terbinafine HCl would have been of the order of 14%. The fact that a solubility of the order of 20% is measured demonstrates the existence of a synergy in the dissolution of terbinafine HCl between propylene glycol and ethanol.

A-2/Water/Ethanol

In water, the visual solubility of the terbinafine HCl is 0.6% w/w.

In the case of a binary water/ethanol mixture (50/50), the visual solubility of the terbinafine HCl is 16.7% w/w, in other words below that obtained with ethanol alone.

A-3/Water/Propylene Glycol

In the case of a binary water/propylene glycol mixture (50/50), the visual solubility of the terbinafine HCl drops to 2.2% w/w, i.e. slightly less than that obtained with propylene glycol alone.

In conclusion, in the binary water/propylene glycol and water/ethanol mixtures, the water lowers the solubility of the terbinafine HCl by comparison, respectively, with the solubilities obtained in propylene glycol and in ethanol.

B/ Ternary Solvent System:

When 15% of water is introduced into a mixture composed either of 70% of propylene glycol and 15% of ethanol, or of 70% of ethanol and 15% of propylene glycol, the visual solubility of the terbinafine HCl goes up, surprisingly, and reaches 25.0% w/w and 25.2% w/w, respectively.

In conclusion, and in contrast to any expectation, the introduction of water (qs 100%) into a binary propylene glycol/ethanol mixture (70/15 or 15/70) increases, surprisingly, the solubility of the terbinafine HCl, although water is a poor solvent per se for terbinafine HCl. There is therefore a synergy, in terms of dissolution, of terbinafine HCl in a ternary propylene glycol/ethanol/water mixture.

In order to come close to optimal conditions for transungual application, namely a large amount of water in the pharmaceutical composition and an amount of terbinafine HCl of at least 10%, the amount of water was then increased to 30% in the presence of equivalent amounts of 35% of propylene glycol and 35% of ethanol.

Under these conditions, the visual solubility of the terbinafine HCl is then 18.5% w/w, i.e. well above 10%, a concentration determined as being optimum in terms of the biological activity of the active principle.

When the amount of water is increased to 33% in the presence of equivalent amounts of propylene glycol (33%) and ethanol (33%), the visual solubility of terbinafine HCl is 15.5% w/w, i.e. still above 10%.

In conclusion, it appears that water, which is desirable for providing moisture to the nail in the intended application, and hence promoting improved spreading of the antifungal agent within the nail matrix, makes it possible to increase the solubility of terbinafine HCl, in spite of its poor solvency for this antifungal agent. This increase in solubility is dependent on the combination propylene glycol and ethanol.

As already seen above, in a binary water/propylene glycol or water/ethanol mixture, the water reduces the solubility of the terbinafine HCl. Therefore, only the combination of a ternary water/propylene glycol/ethanol mixture makes it possible to increase the solubility of terbinafine HCl. It has thus been shown that the solubility of terbinafine HCl is greater than 10% in a ternary water/propylene glycol/ethanol mixture containing at least 30% of water (Graph 1).

The solubility of terbinafine remains greater than 10% with 40% of water, with the proviso that the amount of ethanol is at least greater than or equal to that of the propylene glycol.

It has thus been possible to define the proportions of a ternary propylene glycol/ethanol/water mixture that allow at least 10% of terbinafine HCl to be dissolved, and in which the total amount of water is at least 30%, an amount of water necessary for obtaining improved spreading of the terbinafine within the nail matrix.

2/Compositions Based on the Ternary Solvent System:

The ternary solvent system as defined allowed various formulations to be produced, containing substantial amounts of terbinafine HCl, with no solubility problem. The examples below are given by way of illustration, and are not at all limitative.

For each example, the formula is expressed as a function of the percent of each ingredient used as sold (1st column).

The formula is also expressed by taking account of the water present in each ingredient. In this case, the % corresponding to the ingredients are considered to add up to 100% (2nd column).

The formulations are prepared by following procedure:
Step 1: Dissolve the hydroxytoluene in ethanol.
Step 2: When the Butylhydroxytoluene is fully dissolved, add successively:

### PROPYLENE GLYCOL

### PURIFIED WATER

### EDTA

Stirring until complete solubilization of EDTA.
Step 3: add terbinafine HCL mixture obtained in step 2.
Let stirring until complete solubilization.

### Example 1

| | | Formula (% taking |
|---|---|---|
| | Formula | account of the water |
| Ingredients (w/w) | (% per ingredient) | present in each ingredient) |
| Terbinafine HCl | 10% | 10% |
| Purified water | 34.17% | / |
| Total water (purified | / | 34.17 + (35.53 × |
| water + water present | | 0.05) = 35.95% |
| in the ingredients) | | |
| Propylene glycol | 20.25% | 20.25% |
| Ethanol 100% | / | 35.53 − (35.53 × |
| | | 0.05) = 33.75% |
| Ethanol 95-96% | 35.53% | / |
| Butylated | 0.04% | 0.04% |
| hydroxytoluene | | |
| EDTA | 0.01% | 0.01% |

The physical and chemical stabilities were measured for 3 months at room temperature and at 40° C.

### Physical Stability:

Specifications at T0

Macroscopic appearance: clear, colourless solution

Microscopic appearance: absence of crystals of terbinafine HCl

| | |
|---|---|
| Stability | Time |

| conditions | T 1 month | T 2 months | T 3 months |
|---|---|---|---|
| TA | in line with | in line with | in line with |
| | specifications | specifications | specifications |
| 40° C. | in line with | in line with | in line with |
| | specifications | specifications | specifications |

### Chemical Stability:

| Stability | Time | | |
|---|---|---|---|
| conditions | T 1 month | T 2 months | T 3 months |
| TA | 99.6% | 99.9% | 96.4% |
| 40° C. | 97.2% | 100% | 99.8% |

### Example 2

| | | Formula (% taking |
|---|---|---|
| | Formula | account of the water |
| Ingredients (w/w) | (% per ingredient) | present in each ingredient) |
| Terbinafine HCl | 10% | 10% |
| Purified water | 33.67% | / |
| Total water (purified | / | 33.67 + (35.53 × |
| water + water present | | 0.05) = 35.45% |
| in the ingredients) | | |
| Propylene glycol | 20.25% | 20.25% |
| Ethanol 100% | / | 35.53 − (35.53 × |
| | | 0.05) = 33.75% |
| Ethanol 95-96% | 35.53% | / |
| Hydroxyethylcellulose | 0.50% | 0.50% |
| Butylated | 0.04% | 0.04% |
| hydroxytoluene | | |
| EDTA | 0.01% | 0.01% |

The physical and chemical stabilities were measured for 3 months at room temperature and at 40° C.

### Physical Stability:

Specifications at T0

Macroscopic appearance: clear, colourless, slightly viscous solution

Microscopic appearance: absence of crystals of terbinafine HCl

| | |
|---|---|
| Stability | Time |

| conditions | T 1 month | T 2 months | T 3 months |
|---|---|---|---|
| TA | in line with | in line with | in line with |
| | specifications | specifications | specifications |
| 40° C. | in line with | in line with | in line with |
| | specifications | specifications | specifications |

### Chemical Stability:

| Stability | Time | | |
|---|---|---|---|
| conditions | T 1 month | T 2 months | T 3 months |
| TA | 100.49% | 97.24% | 98.82% |
| 40° C. | 100.1% | 99.8% | 100.1% |

### Example 3

| | | Formula (% taking |
|---|---|---|
| | Formula | account of the water |
| Ingredients (w/w) | (% per ingredient) | present in each ingredient) |
| Terbinafine HCl | 10% | 10% |
| Purified water | 33.44% | / |
| Total water (purified | / | 33.44 + (0.05 × |
| water + water present | | 31.88) = 35.03% |
| in the ingredients) | | |
| Propylene glycol | 19.13% | 19.13% |
| Ethanol 100% | / | 31.88 − (31.88 × |
| | | 0.05) = 30.29% |
| Ethanol 95-96% | 31.88% | / |
| Hydroxyethylcellulose | 0.50% | 0.50% |
| Butylated | 0.04% | 0.04% |
| hydroxytoluene | | |
| EDTA | 0.01% | 0.01% |
| Cyclomethicone | 5% | 5% |

### Example 4

| | | Formula (% taking |
|---|---|---|
| | Formula | account of the water |
| Ingredients (w/w) | (% per ingredient) | present in each ingredient) |
| Terbinafine HCl | 10% | 10% |
| Purified water | 38.75% | / |
| Total water (purified | / | 38.75 + (31.50 × |
| water + water present | | 0.05) = 40.32% |
| in the ingredients) | | |
| Propylene glycol | 18.90% | 18.90% |
| Ethanol 100% | / | 31.50 − (31.5 × |
| | | 0.05) = 29.93% |
| Ethanol 95-96% | 31.50% | / |
| Hydroxyethylcellulose | 0.30% | 0.30% |
| Butylated | 0.04% | 0.04% |
| hydroxytoluene | | |
| EDTA | 0.01% | 0.01% |
| Acetic acid | 0.50% | 0.50% |

### Example 5

| | | Formula (% taking |
|---|---|---|
| | Formula | account of the water |
| Ingredients (w/w) | (% per ingredient) | present in each ingredient) |
| Terbinafine HCl | 10% | 10% |
| Purified water | 33.87% | / |
| Total water (purified | / | 33.87 + (35.53 × |
| water + water present | | 0.05) = 35.65% |
| in the ingredients) | | |
| Propylene glycol | 20.25% | 20.25% |
| Ethanol 100% | / | 35.53− (35.53 × |
| | | 0.05) = 33.75% |
| Ethanol 95-96% | 35.53% | / |
| Hydroxyethylcellulose | 0.30% | 0.30% |
| Butylated | 0.04% | 0.04% |
| hydroxytoluene | | |
| EDTA | 0.01% | 0.01% |

By using the ternary propylene glycol/ethanol/water mixture in certain proportions, we have demonstrated that it is possible to obtain a terbinafine HCl solubility which is much greater than that obtained in each solvent taken separately.
This ternary propylene glycol/ethanol/water mixture therefore exhibits a synergistic effect in the dissolution of terbinafine HCl in the proportions used.
On the basis of this ternary propylene glycol/ethanol/water mixture, it was possible to produce stable compositions containing a minimum of 30% of total water, while permitting solubilization of a terbinafine HCl content of 10% w/w.

1. A pharmaceutical composition comprising:
an antifungal agent selected from the group consisting of allylamines and morpholines, wherein the antifungal agent is present at a concentration of at least 5%; and
a ternary solvent system comprising
water;
at least one branched or straight chain C2-C8 alkanol; and
at least one glycol;
wherein the total amount of water represents more than 30% of the composition.

2. The pharmaceutical composition according to claim 1, wherein the antifungal agent is terbinafine or one of its acid salts.

3. The pharmaceutical composition according to claim 2, wherein the acid salt of terbinafine is terbinafine hydrochloride.

4. The pharmaceutical composition according to claim 1, wherein the concentration of antifungal agent is greater than or equal to 8%.

5. The pharmaceutical composition according to claim 1, wherein the alkanol is ethanol.

6. The pharmaceutical composition according to claim 1, wherein the glycol is propylene glycol.

7. The pharmaceutical composition according to claim 1, wherein the composition takes the form of a lotion, a spray, an emulsion, a foam or a gel.

8. The pharmaceutical composition according to claim 1, wherein the composition further comprises at least one compound selected from the group consisting of a chelating agent, an antioxidant, a polymer, an antiseptic agent, and an emollient.

9. A method of making a medicament, the method comprising making the medicament so that it comprises a composition according to claim 1 in an amount effective for application of the medicament to a nail of an individual subject in need for treating onychomycos.

10. The composition according to claim 4, wherein the concentration of antifungal agent is greater than or equal to 10%.
